(19) United States
(12) Patent Application Publication
(10) Pub. No.: US 2011/0257642 A1
(43) Pub. Date: Oct. 20, 2011

(54) Method for producing a permanent or nearly permanent skin image, design or tattoo by freezing the skin

(76) Inventor: Charles Sherman Griggs, III, Land O Lakes, FL (US)

(21) Appl. No.: 12/799,035

(22) Filed: Apr. 16, 2010

(51) Int. Cl. A61B 18/02

(52) U.S. Cl. 606/22; 606/20

(57) ABSTRACT

This invention provides a relatively fast and safe method of producing a permanent or nearly permanent skin image, design, or tattoo similar in appearance to that of a solar tattoo. A protective design template is placed on the skin and a cryogenic agent applied to freeze exposed portions of the skin, producing hypopigmentation. An image is defined by the contrast between the treated (hypopigmented) skin and the adjacent untreated skin.

### CROSS-REFERENCES TO RELATED APPLICATIONS

Not applicable.

### STATEMENT REGARDING FEDERALLY SPONSORED RESEARCH OR DEVELOPMENT

Not applicable.

### MICROFICHE APPENDIX

Not applicable.

### BACKGROUND OF THE INVENTION

1. Field of the Invention

This invention relates to the field of means of creating an ornamental image, design or tattoo on a portion of the human skin.

2. Description of Related Art

Various devices and methods have developed to satisfy the need for decorating or adorning the human body with permanent or temporary tattoos. Perhaps the most common method of tattooing today is carried out using a needle to insert indelible ink or dye into the layers of human skin to change its pigment, thereby enabling the body to be decoratively modified. Traditional tattooing provides permanence sought by many, but it is a somewhat painful procedure and carries with it the risk of possible infection, even if carried out properly by a licensed tattoo artist. The designs produced by this method of tattooing typically change the pigment by making it darker, rather than lighter.

In contrast to the traditional needle and dye method, solar tattooing provides a means of creating designs on the human skin through the skin's protective capacity to tan in response to sunlight or other sources of ultraviolet (UV) radiation. Tanning is a natural process whereby the skin produces brown-colored pigment, known as melanin, in response to natural or artificial UV radiation. Solar tattooing is accomplished by placing a decorative template, such as a sticker, on the skin, and then exposing those areas of skin not covered by the decorative template to UV radiation from the sun or some other source. The areas of the skin not covered by the decorative template tan, while the areas of skin covered and protected by the decorative template do not.

When the decorative template is removed, a decoration appears on the skin from the contrast between those areas that have tanned and those areas that have not. This process can be further carried out by the strategic application of sunscreen in lieu of, or in addition to, decorative templates to control where melanin concentrates in the skin.

The chief disadvantage of solar tattoos is that they are temporary and fade as the surrounding skin gradually becomes less tan, or as the area of skin originally covered and protected by the decorative template or sunscreen becomes exposed to natural or artificial UV radiation and itself begins to tan in response to that radiation. Solar tattoos are also difficult or impossible to create on skin that is already tan or naturally dark. Moreover, they require sustained exposure to the sun or to an artificial source of UV radiation, which can promote skin cancers.

In cryosurgery, cryogenic agents are commonly employed to selectively destroy through cooling biological tissue, namely, abnormal cells giving rise to external skin lesions that may present cancerous, precancerous, or non-cancerous conditions. One consequence of the process of freezing the skin is hypopigmentation, or the loss of skin color caused by melanocyte or melanin depletion. Hypopigmentation is caused by the sensitivity of melanocytes to freezing, and it becomes more likely with longer freeze times. Hypopigmentation typically occurs over a period of days or weeks following application of a cryogenic agent.

### BRIEF SUMMARY OF THE INVENTION

The instant invention presents a method of creating a permanent or nearly permanent design on the human skin through the application of a cryogenic agent and a protective decorative template.

### BRIEF DESCRIPTION OF THE SEVERAL VIEWS OF THE DRAWINGS

FIG. 1 is a perspective view of a protective decorative template with a cut-out design of a star applied to an area of the skin.

FIG. 2 is a perspective view of a cryogenic agent being applied to areas of the skin exposed within the inner borders of said protective decorative template.

FIG. 3 is a perspective view of said protective decorative template being removed after the cryogenic agent has been applied.

FIG. 4 is a perspective view of the resulting tattoo design formed by the contrast between treated (hypopigmented) skin and untreated skin within days or weeks following the application of the cryogenic agent to the skin exposed within the inner borders of said protective decorative template.

FIG. 5 is a perspective view of a cryogenic agent being applied to exposed areas of the skin surrounding a protective decorative template in the shape of a star without any cut-out portions.

FIG. 6 is a perspective view of a tattoo design formed using the protective decorative template referenced in FIG. 5.

### REFERENCE NUMERALS IN THE DRAWINGS

1 Untreated skin
2 Protective decorative template
3 Inner border
4 Outer border
5 Cryogenic sprayer
6 Cryogenic agent
7 Treated (hypopigmented) skin

### DETAILED DESCRIPTION OF THE INVENTION

This invention provides a relatively fast and safe method of producing a permanent or nearly permanent skin image, design, or tattoo without the use of inks, dyes, or needles. The image produced by this method is similar in appearance to that of a solar tattoo. It may be produced using any form of cryogenic agent, such as liquid nitrogen or argon gas, used to freeze the human skin. Techniques for applying the cryogenic agent may include any commonly used method, such as liquid sprays, cotton-tip applicators, or cryoprobes.

The cryogenic agent is used in conjunction with a protective decorative template, such as a stencil or sticker. The protective decorative template may be in any shape, and may or may not have a cut-out shape or collection of shapes in its central portion. The protective decorative template functions to cover and protect portions of the skin from being frozen by the cryogenic agent, and informs the design to be achieved. Depending on the effect and design desired and the nature of the protective decorative template, the cryogenic agent can be applied to the exposed skin surrounding the outer border of the template or to the skin exposed by any inner border formed by a cut-out shape in the central portion of the template, or both.

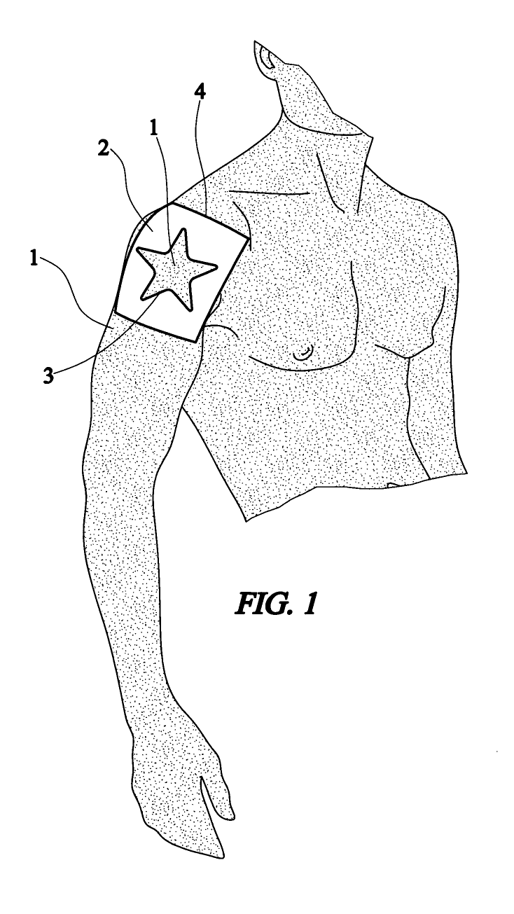

As illustrated in FIG. 1, a protective decorative template 2, which as in this embodiment may be a sticker designed to adhere removeably to human skin, is applied to the area of the skin where the tattoo is desired to be located. The illustrated protective decorative template 2 has an inner border 3 and an outer border 4. The inner border 3, formed by a cut-out from the central portion of the protective decorative template 2, is in the shape of a star in this illustration, but any form or number of decorative shapes could be fashioned in the center portion of the protective decorative template 2, and its outer border 4 could be in any given shape.

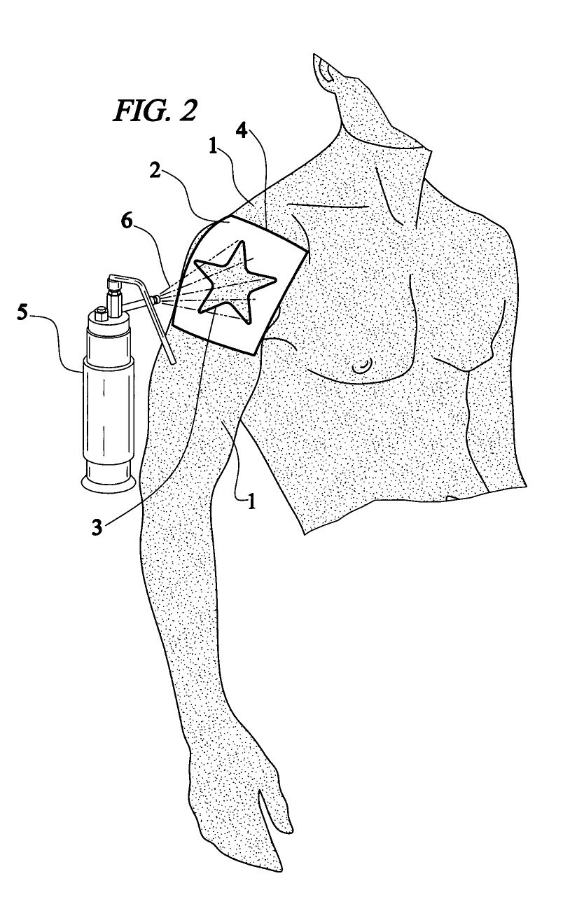

In the illustrated embodiment, the portion of the skin to be lightened through hypopigmentation is solely that portion of skin exposed within the inner border 3 of the decorative template 2. In FIG. 2, a cryogenic sprayer 5, such as the Cry-AC® liquid nitrogen sprayer produced by Brymill Cryogenic Systems, is used to apply the cryogenic agent 6 to that portion of the skin exposed within the inner border 3 of the decorative template 2.

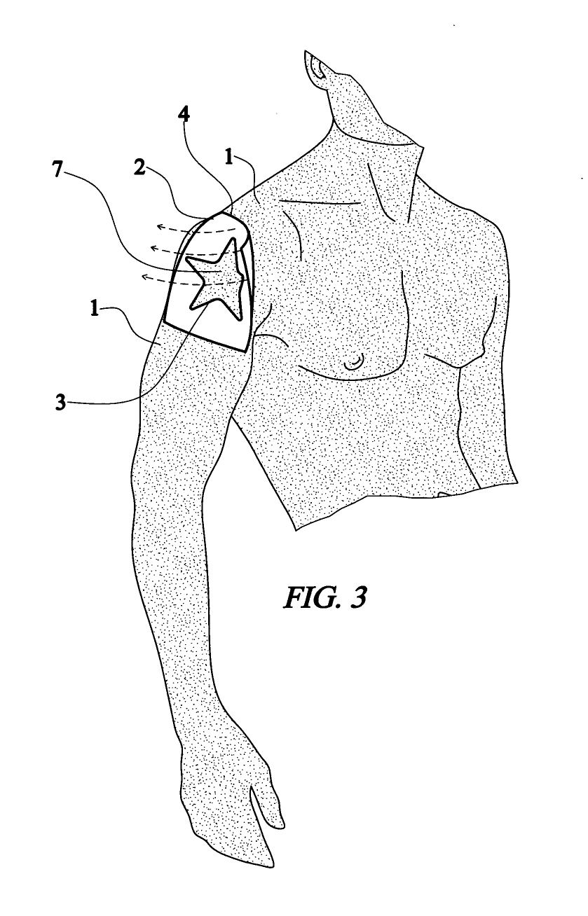

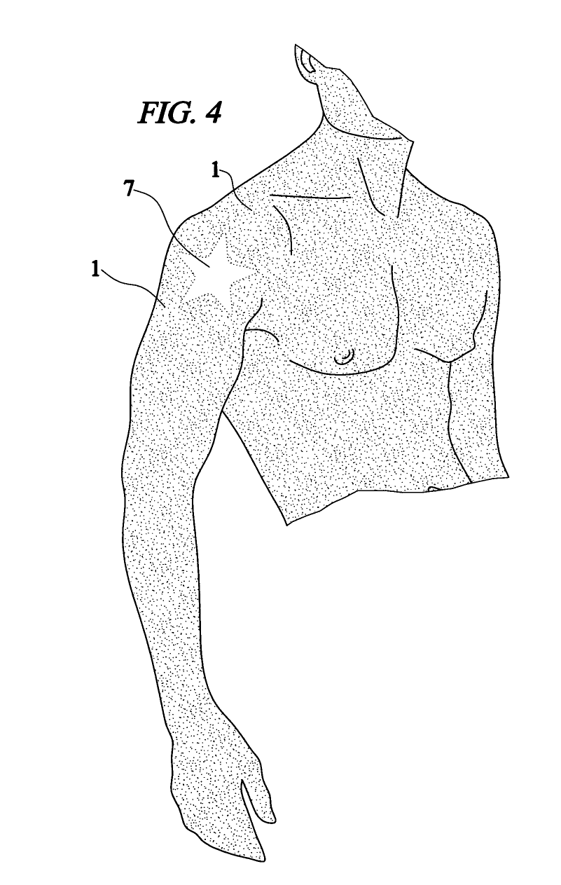

FIG. 3 illustrates the removal of the protective decorative template 2 following the application of the cryogenic agent 6. FIG. 4 illustrates the area of treated (hypopigmented) skin 7 and the resulting design or tattoo formed by the contrast between the treated (hypopigmented) skin 7 and the adjacent untreated skin 1, which develops within days or weeks following the application of the cryogenic agent 6.

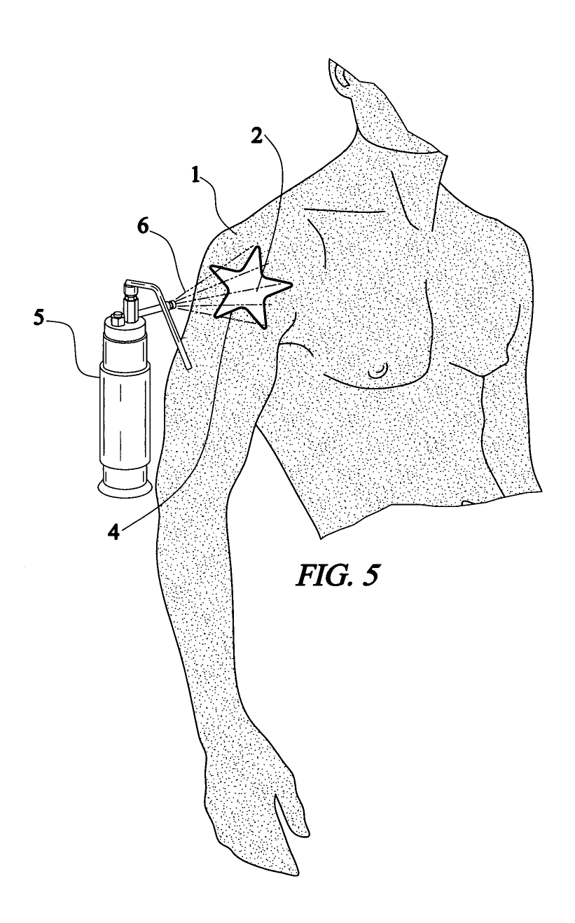

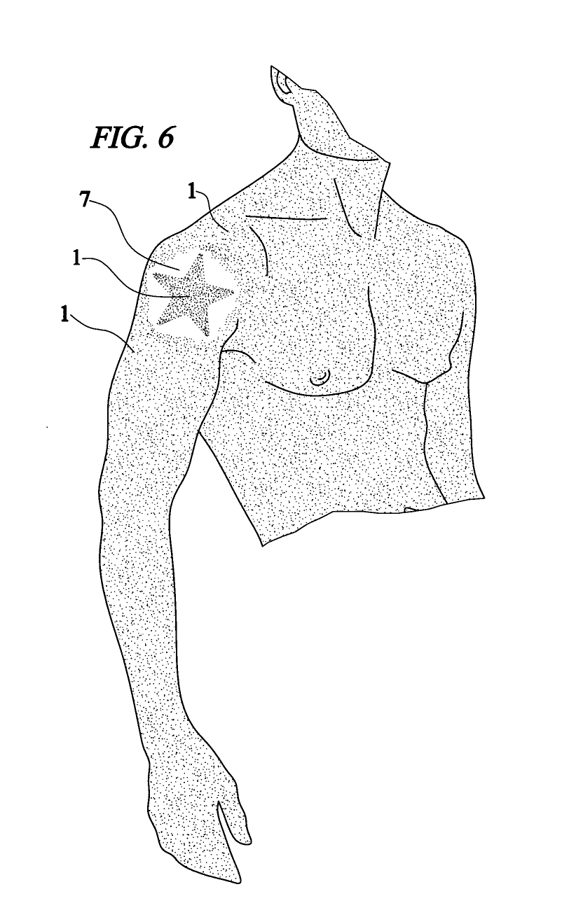

FIG. 5 illustrates the application of a cryogenic agent 6 around the outer border 4 of a protective decorative template 2 that has no cut-outs. The outer border 4 itself is in the shape of a star. The resulting design or tattoo formed by the contrast between the treated (hypopigmented) skin 7 and the adjacent untreated skin 1 is reflected in FIG. 6.

1. A method of creating an image on the human skin, comprising the steps of:
placing a protective decorative template on an area of the human skin;
applying a cryogenic agent to areas of the skin not covered by the protective decorative template;
removing the protective decorative template from the skin.

2. The method of claim 1, in which the protective decorative template is a removeably adhesive sticker.

3. The method of claim 1, in which the protective decorative template has one or more cut-out shapes in the central portion of the template.

4. The method of claim 3, in which the cryogenic agent is applied solely to areas of the skin exposed by the one or more cut-out shapes in the central portion of the template.

5. The method of claim 1, in which the protective decorative template has no cut-out shapes in the central portion of the template and the cryogenic agent is applied to exposed areas of the skin immediately adjacent said protective decorative template.

6. The method of claim 1, in which the cryogenic agent is liquid nitrogen.

7. The method of claim 1, in which the cryogenic agent is argon.

8. The method of claim 1, in which the cryogenic agent is applied with a sprayer.

9. The method of claim 1, in which the cryogenic agent is applied with a cotton-tip applicator.

10. The method of claim 1, in which the cryogenic agent is applied with a cryoprobe.
